(19) United States
(12) Patent Application Publication
(10) Pub. No.: US 2016/0183863 A1
(43) Pub. Date: Jun. 30, 2016

(54) PHYSIOLOGICAL DETECTING DEVICE

(71) Applicant: YONGLIN BIOTECH CORP., New Taipei City (TW)

(72) Inventors: Jiann-Hua Wang, New Taipei City (TW); Chen-Yu Lin, New Taipei City (TW)

(21) Appl. No.: 14/972,415

(22) Filed: Dec. 17, 2015

Related U.S. Application Data
(60) Provisional application No. 62/098,692, filed on Dec. 31, 2014.

(30) Foreign Application Priority Data
Apr. 15, 2015 (TW) 104205659

(51) Int. Cl. A61B 5/00; A61B 10/00; A61B 5/145

(52) U.S. Cl. CPC A61B 5/4343; A61B 5/14546; A61B 10/007; A61B 2562/0295

(57) ABSTRACT

A physiological detecting device includes a measuring unit having a test strip end, a test strip reaction zone and a connecting end. The test strip end includes at least one test strip for taking a sample of a test solution containing at least one physiological hormone. The test strip reaction zone reacts with at least one physiological hormone detected on the test strip end. The engaging portion is engaged to the connecting end of the measuring unit in the first instance, and is also engaged to the test strip end of the measuring unit in the second instance; whereby, in the second instance, the engaging portion is engaged to the test strip end of the measuring unit, so as to push the test solution sampled on the test strip end to the test strip reaction zone for the reaction of the test strip to be performed.

### CROSS REFERENCE TO RELATED APPLICATIONS

This application claims priority benefits of U.S. Provisional Patent Application No. 62/098,692 filed on Dec. 31, 2014 and Taiwanese Patent Application No. 104205659 filed on Apr.15, 2015, which are hereby incorporated by reference in their entirety.

### BACKGROUND OF THE INVENTION

1. Field of the Invention

The present invention relates to a detecting device, and relates particularly to a detecting device that is used for measuring physiological values.

2. The Prior Arts

In the present day, modern people are often busy with work. As such, the average age of marriage increases continuously, and the plans of starting a family are often bothersome to newly married couples who are frequently busy. Women from the traditional Chinese societies face greater pressure compared to men when it comes to having children. Therefore, how women control the ovulation period which is key to pregnancy has become very important. Accordingly, ovulation test strips or basic thermometers are provided by the market, in order to help women control the date of conception.

Generally, during the menstrual cycle of women, the level of luteinizing hormone (LH) begins to increase, reaches a peak on the day before ovulation, and the level of LH decreases rapidly after ovulation. The follicle-stimulating hormone (FSH) will reach a high point before ovulation, after which the level of FSH will begin to decrease; the level of FSH will reach a low point around three days before ovulation, and the level of FSH reaches another high point on the day before ovulation. In terms of basic body temperature, the basic body temperature of the general adult woman decreases by 0.1-0.2° C. before ovulation, and the basic body temperature increases by 0.3-0.5° C. after ovulation. A temperature difference of 0.4-0.7° C. exists before and after ovulation. Accordingly, LH agents, FSH agents and basic thermometers are provided in the market for the adult women. However, no matter whether it is the LH agent, FSH agent or the basic thermometer, there are still disadvantages associated with these.

The LH agent is used for detecting the changes in the level of LH, but as shown in FIG. 1, large changes in LH levels can only be seen on the day before ovulation, and LH detection cannot be clearly seen at other times besides the day before ovulation. When the levels of LH detected are significantly increased, the amount of time left before ovulation is less than 24 hours, and such short amount of time is not appropriate for the preparatory work which are required before pregnancy can take place.

The levels of FSH that are detected by the FSH agent will have two high points before ovulation. However, the difference between the high point and the low point in the FSH levels is not as obvious as the changes in the levels of the LH. As such, there are difficulties when interpreting the date of ovulation, and difficulties are also present when making judgments on the day of ovulation.

The basic thermometer makes use of the temperature difference of 0.4-0.7° C. at both before and after ovulation to calculate the date of ovulation. However, since the human body temperature changes in accordance with conditions of the body, such as in situations when the person has a cold or fever, the basic temperature of the body also changes accordingly. As such, if the ovulation date is judged only by using the method of measuring the basic body temperature, then this judgment is associated with errors.

### SUMMARY OF THE INVENTION

One of the objectives of the present invention is to provide a physiological detecting device for the detection of at least one physiological hormone, in order to increase the degree of accuracy while predicting the time of ovulation during the menstrual cycle of women. The physiological detecting device of the present invention includes a measuring unit, and may include a test strip end, a test strip reaction zone and a connecting end; whereby the test strip end may include at least one test strip for taking a sample of a test solution containing at least one physiological hormone. The test strip reaction zone may react with the at least one physiological hormone that is on the test strip end. The engaging portion may be engaged to the connecting end of the measuring unit in a first instance, and may be engaged to the test strip end of the measuring unit in a second instance. In the second instance, the engaging portion may be engaged to the test strip end of the measuring unit, for pushing the test solution sampled on the test strip reaction zone for the test strip reaction to be performed.

Preferably, the first instance may refer to the time that the physiological detecting device is beginning to be used, and the second instance may refer to the time needed for the physiological detecting device to detect the test solution.

Preferably, the test solution may be urine from a human body.

Preferably, the engaging portion may include a handheld anti-slip portion, whereby the handheld anti-slip portion may be an anti-slip handle.

Preferably, the test strip end may include a first test strip and a second test strip, to correspondingly detect a first physiological hormone and a second physiological hormone.

Preferably, the test strip reaction zone may include a first detecting line, a second detecting line and a reference line; the first detecting line may react with the first physiological hormone that is detected on the first test strip, the second detecting line may react with the second physiological hormone that is detected on the second test strip.

Preferably, the first detecting line and the second detecting line may present a negative reaction or a positive reaction respectively corresponding to the first physiological hormone and the second physiological hormone.

Preferably, the first physiological hormone may be Follicle-stimulating hormone (FSH), and the first test strip may be FSH-test strip.

Preferably, the second physiological hormone may be Luteinizing hormone (LH), and the second test strip may be LH-test strip.

Preferably, the test strip reaction zone may be covered by a plastic housing.

### BRIEF DESCRIPTION OF THE DRAWINGS

The present invention can be understood in more detail by reading the subsequent detailed description in conjunction with the examples and references made to the accompanying drawings, wherein:

FIG. 1 is a schematic diagram showing the physiological detecting device in accordance with an embodiment of the present invention;

FIG. 2 is a schematic diagram showing the use of the physiological detecting device in the first instance in accordance with an embodiment of the present invention; and

FIG. 3 is a schematic diagram showing the use of the physiological detecting device in the second instance in accordance with an embodiment of the present invention.

### DETAILED DESCRIPTION OF THE PREFERRED EMBODIMENT

The present invention may be embodied in various forms and the details of the preferred embodiments of the present invention will be described in the subsequent content with reference to the accompanying drawings. The drawings (not to scale) depict only the preferred embodiments of the invention and shall not be considered as limitations to the scope of the present invention. Modifications of the shape of the present invention shall be considered within the spirit of the present invention.

It should be noted that the structures, proportions, sizes and the like of the drawings of the current specification are only for depicting the disclosures of the present invention, to enable easy reading and understanding by persons skilled in the art, and these are not meant to be conditions that limit the present invention. As such, the drawings are not technologically meaningful. Any modifications of the structure, proportion relationships or any adjustments of the sizes should fall within the scope that is covered by the technical content of the present invention, without affecting the effects produced by the present invention and without affecting the goals of the present invention.

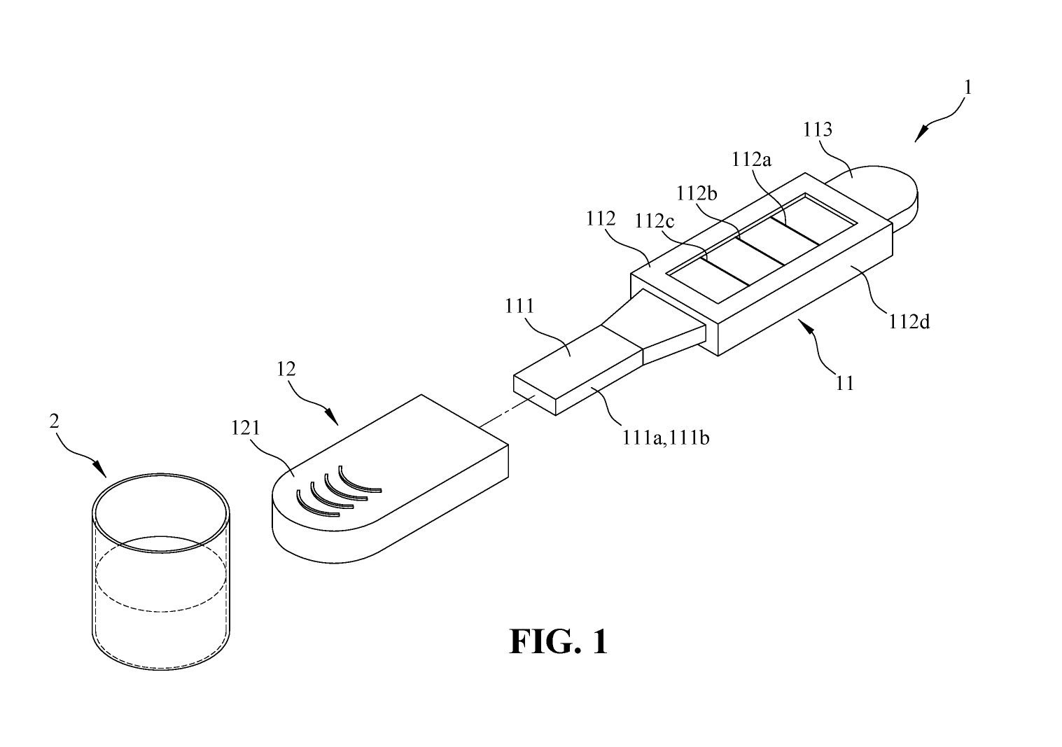

First of all, referring to FIG. 1, FIG. 1 is a schematic diagram showing the physiological detecting device of the present invention. The physiological detecting device 1 of the present invention may include: a measuring unit 11, which may include a test strip end 111, a test strip reaction zone 112 and a connecting end 113. The test strip end 111 may include at least a test strip, for taking a sample of a test solution 2 that contains at least a physiological hormone. The test strip reaction zone 112 may react with at least a physiological hormone that is detected by the test strip end. Moreover, the engaging portion 12 may be engaged to the connecting end 113 of the measuring unit 11 in the first instance, and may be engaged to the test strip end 111 of the measuring unit 11 in the second instance; whereby, in the second instance, the engaging portion 12 may be engaged to the test strip end 111 of the measuring unit 11, so as to push the test solution 2 sampled on the test strip 111 to the test strip reaction zone 112 for the test strip reaction to be performed.

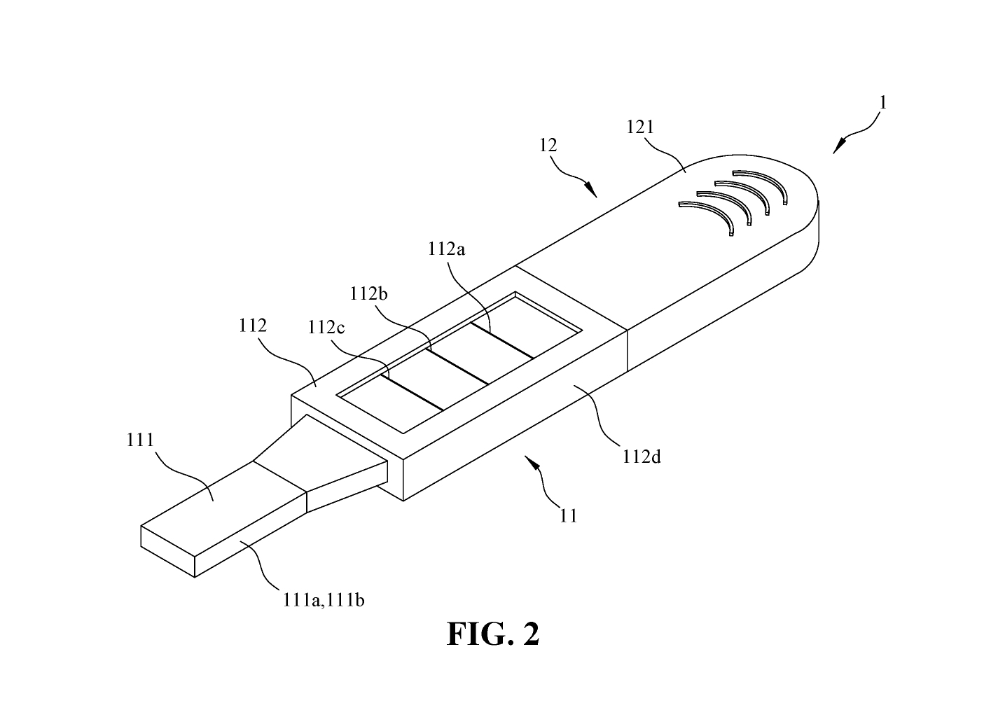

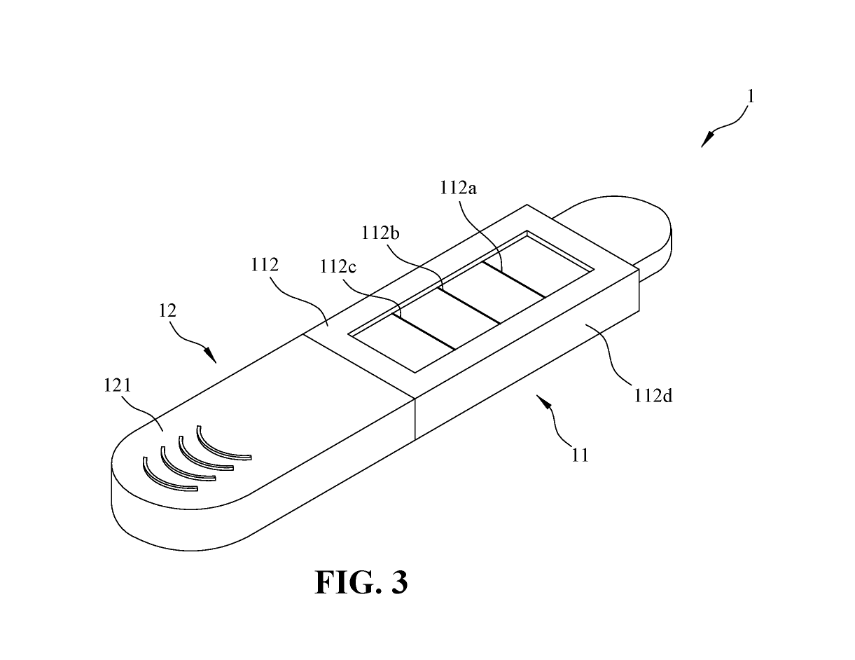

In addition, as shown in FIG. 2, FIG. 2 is a schematic diagram showing that the engaging portion 12 may be engaged to the connecting end 113 of the measuring unit in the first instance. FIG. 3 is a schematic diagram showing that the engaging portion 12 may be engaged to the test strip end 111 of the measuring unit 11 in the second instance.

In accordance with a preferred exemplary embodiment of the present invention, and referring to FIG. 1 to FIG. 3, the first instance may indicate the time that the physiological detecting system is beginning to be used, and the second instance may indicate the time that the test solution 2 is detected by the physiological detecting device.

In accordance with a preferred exemplary embodiment of the present invention, the engaging portion 12 may include a handheld anti-slip part 121, which is to enable the handle to have an anti-slip function, but is not limited to this.

In accordance with a preferred exemplary embodiment of the present invention, the test solution 2 of the present invention may be urine from a human body.

In accordance with a preferred exemplary embodiment of the present invention, the test strip end 111 may include a first test strip 111a and a second test strip 111b, which may be for correspondingly detecting the first physiological hormone and the second physiological hormone. The test strip reaction zone 112 may include a first detecting line 112a, a second detecting line 112b, as well as a reference line 112c. The first detecting line 112a may react with the first physiological hormone that is detected by the first test strip 111a, the second detecting line 112b may react with the second physiological hormone that is detected by the second test strip 111b. The first detecting line 112a and the second detecting line 112b may present a negative reaction or a positive reaction respectively corresponding to the first physiological hormone and the second physiological hormone detected.

In accordance with a preferred exemplary embodiment of the present invention, the first physiological hormone may be Follicle-stimulating hormone, FSH; the first test strip 111a may be FSH-test strip; the second physiological hormone may be Luteinizing hormone, LH; and the second test strip 111b may be LH-test strip.

Whereby, the test strip reaction zone 112 may be covered by a plastic housing, but is not limited to this.

Although the present invention has been described with reference to the preferred embodiments, it will be understood that the invention is not limited to the details described thereof. Various substitutions and modifications have been suggested in the foregoing description, and others will occur to those of ordinary skill in the art. Therefore, all such substitutions and modifications are intended to be embraced within the scope of the invention as defined in the appended claims.

What is claimed is:

1. A physiological detecting device, comprising:
a measuring unit, comprising a test strip end, a test strip reaction zone and a connecting end, wherein the test strip end comprises at least one test strip for taking a sample of a test solution containing at least one physiological hormone, and the test strip reaction zone reacts with the at least one physiological hormone that is on the test strip end; and
an engaging portion, engaged to the connecting end of the measuring unit in a first instance, and engaged to the test strip end of the measuring unit in a second instance;
wherein in the second instance, the engaging portion is engaged to the test strip end of the measuring unit, for pushing the test solution sampled on the test strip reaction zone for a test strip reaction to be performed.

2. The physiological detecting device of claim 1, wherein the first instance refers to the time that the physiological detecting device is beginning to be used, and the second instance refers to the time needed for the physiological detecting device to detect the test solution.

3. The physiological detecting device of claim 1, wherein the test solution is urine from a human body.

4. The physiological detecting device of claim 1, wherein the engaging portion comprises a handheld anti-slip portion.

5. The physiological detecting device of claim 1, wherein the test strip end comprises a first test strip and a second test strip, to correspondingly detect a first physiological hormone and a second physiological hormone.

6. The physiological detecting device of claim 1, wherein the test strip reaction zone comprises a first detecting line, a second detecting line and a reference line, the first detecting line reacts with the first physiological hormone that is detected on the first test strip, the second detecting line reacts with the second physiological hormone that is detected on the second test strip.

7. The physiological detecting device of claim 6, wherein the first detecting line and the second detecting line present a negative reaction or a positive reaction respectively corresponding to the first physiological hormone and the second physiological hormone.

8. The physiological detecting device of claim 5, wherein the first physiological hormone is Follicle-stimulating hormone (FSH), and the first test strip is FSH-test strip.

9. The physiological detecting device of claim 5, wherein the second physiological hormone is Luteinizing hormone (LH), and the second test strip is LH-test strip.

10. The physiological detecting device of claim 1, wherein the test strip reaction zone is covered by a plastic housing.
